(19) United States
(12) Patent Application Publication
(10) Pub. No.: US 2015/0037856 A1
(43) Pub. Date: Feb. 5, 2015

(54) Rapid and low cost enzymatic full conversion of lignocellulosic biomass

(71) Applicants: Jan Larsen, Tommerup (DK); Martin Dan Jeppesen, Odense V (DK)

(72) Inventors: Jan Larsen, Tommerup (DK); Martin Dan Jeppesen, Odense V (DK)

(73) Assignee: Inbicon A/S, Fredericia (DK)

(21) Appl. No.: 14/350,081

(22) PCT Filed: Oct. 6, 2011
(86) PCT No.: PCT/IB2011/054406

§ 371 Date: Aug. 28, 2014

(51) Int. Cl. C12P 7/14

(52) U.S. Cl. CPC C12P 7/14
USPC 435/162

(57) ABSTRACT

Methods are provided for improved processing of lignocellulosic biomass. Hydrothermally pretreated lignocellulosic biomass is subject to separate hydrolysis and fermentation (SHF) or prehydrolysed and subject to simultaneous saccharification and fermentation (SSF) at high initial loadings of cellulase enzymes, at least 15 FPU/g DM. The cellulase enzymes are subsequently recycled and used in subsequent hydrolysis cycles along with a lower dose supplementation of fresh enzyme. Loss of enzyme activity between hydrolysis cycles is offset by improved overall process advantage.

### FIELD OF THE INVENTION

The invention relates, in general, to methods of processing lignocellulosic biomass including separate hydrolysis and fermentation (SHF) and simultaneous saccharification and fermentation (SSF) of pretreated biomass, and in particular, to improvements of process efficiency by enzymatic hydrolysis and cellulase recycling at very high initial cellulase enzyme loading.

### BACKGROUND

Lignocellulosic biomass offers a promising alternative to petroleum, providing renewable and “carbon neutral” sources of fuels, such as bioethanol, and of other traditionally petroleum-based products such as plastics. Lignocellulosic biomass can be enzymatically hydrolysed to provide fermentable carbohydrates that are in turn useful in a variety of biosynthetic processes.

Because of its complex chemical structure, lignocellulose can usually only be efficiently hydrolysed by presently known enzyme activities after some pre-treatment that renders cellulose fibers accessible to enzyme catalysis. Such pre-treatment processes typically involve heating to comparatively high temperatures, between 100 and 250° C. An intense interest has arisen in methods of biomass pre-treatment and processing that reduce costs or otherwise increase commercial viability on production scale.

One factor which heavily influences overall costs of lignocellulosic biomass processing is the cost of cellulase enzymes. Methods of enzymatic hydrolysis of pretreated lignocellulosic biomass that utilize cellulase enzymes more efficiently or that improve overall yields of fermentable carbohydrates are, accordingly, advantageous.

One category of improvements that has received considerable attention has been strategies for recycling enzymes or otherwise reducing enzyme consumption.

Previous studies on cellulase recycling indicated that recovery depends primarily on two factors—(i) degree of hydrolysis and (ii) lignin content of the feedstock used. As a general trend, enzyme recovery in recycling was reportedly improved where feedstocks were more completely hydrolysed. However, this tendency for improved cellulase recovery—as feedstocks became more completely hydrolysed—was offset by a related tendency for increased non-specific binding of cellulases to lignin. A comparison of different recycling strategies indicated that lignin content of the feedstock was the single most important variable controlling cellulase recovery. See ref. 1.

A number of cellulase recycling strategies have been proposed. In feedstocks which were not de-lignified by chemical treatments, the prevailing approach has been to recover enzymes at an intermediate degree of feedstock hydrolysis. Cellulosic residues were first filtered or otherwise recovered from hydrolysis reaction mixtures, then contacted with fresh feedstocks, permitting recovery of cellulase enzyme activities at levels as high as 50-70%. See ref. 2 and ref. 3.

In feedstocks which were de-lignified by chemical treatments, older studies suggested that maximal enzyme recycling could be obtained after “complete” hydrolysis, in which no cellulosic residue remains. See ref. 1. However, more recent studies indicate that, using de-lignified substrates combined with surfactants, optimal cellulase recycling requires recovery from both supernatant and cellulosic residue, i.e., that optimum recovery requires a degree of feedstock hydrolysis that is less than “complete.” See ref. 4 and 5.

Previous efforts to develop cellulase recycling strategies have focused on reducing enzyme loading. Dilute enzyme regimes have generally been considered desirable. The extent to which any given feedstock is hydrolysed at any given time point in the enzymatic hydrolysis reaction is increased at higher enzyme loading. See ref. 6.

However, this effect is logarithmic, meaning that large differences in enzyme loading are required to achieve comparatively small differences in percent conversion at a given time point. Consequently, in order to optimize enzymatic hydrolysis times without incurring excessive enzyme costs, the art has previously considered a comparatively dilute enzyme concentration to be desirable. See ref. 7. Technoeconomic modeling of commercial scale bioethanol production previously indicated that cellulase loading on the order of 10 FPU/g dry matter (DM) pretreated biomass was ideal, since this was believed to provide the highest glucose yield within a reasonable hydrolysis time for a reasonable cost. See ref. 3.

Cellulase loadings of >10 FPU/g DM pretreated biomass have not previously been considered desirable in commercial scale bioethanol production. One extensive comparison of overall costs and glucose yields in a continuous-hydrolysis recycling system indicated that a high enzyme loading of 20 FPU/g DM provided no advantage over a more dilute loading of 10 FPU/g DM, using feedstocks that were not de-lignified. See ref. 2.

We have discovered that cellulase recycling in commercial bioethanol production that relies on SHF or SSF can be conducted with overall advantage by using high initial enzyme loading, at least 17 FPU/g DM, followed by lower dose enzyme supplementation on each hydrolysis cycle of cellulase activity recovered from the previous cycle. At these high cellulase levels non-specific lignin-binding apparently becomes saturated. High enzyme dose levels can be maintained over multiple hydrolysis cycles using only low level supplementation with fresh enzyme. These high cellulase activity levels greatly reduce hydrolysis times, leading to reduced capital costs and increased capacity in production scale. High cellulase activity levels also result in more complete % conversion, reducing biomass costs per liter ethanol produced. Effective results are obtained using pretreated lignocellulosic feedstocks that were not de-lignified. In some cases, hydrolysis yield can be so much improved and cellulase activity recycled to such high degree that final enzyme consumption per liter ethanol is reduced compared with that achieved at the low enzyme levels recommended by commercial enzyme suppliers.

### SUMMARY

Methods are provided for improved processing of lignocellulosic biomass in bioethanol production. Hydrothermally pretreated lignocellulosic biomass is subject to separate hydrolysis and fermentation (SHF) or prehydrolysed and subject to simultaneous saccharification and fermentation (SSF) at high initial loadings of cellulase enzymes, at least 15 FPU/g DM. The cellulase enzymes are subsequently recycled and used in subsequent hydrolysis cycles along with a lower dose supplementation of fresh enzyme. Loss of enzyme activity between hydrolysis cycles is offset by improved overall process advantage.

### BRIEF DESCRIPTION OF THE FIGURES

FIG. 1 shows ethanol yield as a function of time and cellulase dose in experiments with a 72 hours SSF process regime.

FIG. 2 shows glucose concentration after 6 hours hydrolysis as a function of cellulase dose expressed as FPU/g DM.

FIG. 3 shows the scheme of experiments reported in example 3.

FIG. 4 shows ethanol concentration achieved after each recycling cycle, using a commercially available cellulase preparation optimized for lignocellulosic biomass conversion and provided by NOVOZYMES™ to practice methods of the invention.

FIG. 5 shows glucose concentration after 6 hours prehydrolysis demonstrating cellulase activity recovery after each recycling cycle, using a commercially available cellulase preparation optimized for lignocellulosic biomass conversion and provided by GENENCOR™ to practice methods of the invention.

### DETAILED DESCRIPTION OF SOME EMBODIMENTS

Provided are methods for processing lignocellulosic biomass in bioethanol production with improved efficiency.

The largest commercial cellulase enzyme producers have been working intensively since 2000 to decrease production cost and improve specific activities of cellulase enzyme mixtures optimized for conversion of lignocellulosic biomass in second generation bioethanol production. NOVOZYMES™ and GENENCOR™ in particular launched new commercial cellulase enzyme mixtures in 2009-2010 under the trademarks CELLIC CTEC2™ and ACCELLERASE 1500™ respectively.

GENENCOR™ in 2009 launched an improved variant of a commercially available biomass enzyme developed specifically for second generation biorefineries and sold under the trademark ACCELLERASE 1500™. It has been shown to successfully hydrolyze a range of pretreated feedstocks including sugar cane bagasse, corn stover, wheat straw, and softwood pulp. The product information sheet for ACCELLERASE 1500™ indicates a dosage optimization range of 0.1-0.5 mL per g cellulose or roughly 0.05 to 0.25 mL per g DM pretreated biomass.

NOVOZYMES™ in 2010 launched an improved variant of a commercially available biomass enzyme developed specifically for second generation biorefineries and sold under the trademark CELLIC CTEC2™. It has been shown to successfully hydrolyze a range of pretreated feedstocks including sugar cane bagasse, corn stover, wheat straw, and softwood pulp. Product information materials distributed by NOVOZYMES™, specifically the “dosing guidelines” for CELLIC CTEC2™ given in “Fuel Ethanol Application” materials, indicates a range of doses from low to high, corresponding to a “target for commercially feasible cellulose hydrolysis” within the range of 0.015-0.06 g enzyme per g cellulose or roughly 0.0075 to 0.03 g enzyme per g DM pretreated biomass.

“Cellulase activity” refers to enzymatic hydrolysis of 1,4-β-D-glycosidic linkages in cellulose. In commercial or other cellulase enzyme preparations obtained from bacterial, fungal or other sources, cellulase activity typically comprises a mixture of different enzyme activities, including endoglucanases and exoglucanases (also termed cellobiohydrolases), which respectively catalyse endo- and exo-hydrolysis of 1,4-β-D-glycosidic linkages, along with β-glucosidases, which hydrolyse the oligosaccharide products of exoglucanase hydrolysis to monosaccharides. Commercial mixtures optimized for hydrolysis of lignocellulosic biomass also often contain enzymes that are useful in conversion of lignocellulosic biomass but that are not cellulases per se, such as hemicellulases, which catalyse hydrolysis of the heteropolymer hemicellulose that is associated with cellulose in lignocellulosic materials, and that is comprised of a variety of monomer sugars most notably xylose, but also including mannose, galactose, rhamnose, arabinose, and other sugars.

As is well known in the art, total cellulase activity, including any mixture of different cellulase enzymes, can be conveniently measured as a single activity expression termed “filter paper units.” As used herein, the term “filter paper units” (FPU) refers to filter paper units as determined by the method of Adney, B. and Baker, J., Laboratory Analytical Procedure #006, “Measurement of cellulase activity”, Aug. 12, 1996, the USA National Renewable Energy Laboratory (NREL), which is expressly incorporated by reference herein in entirety. It will be readily understood by those skilled in the art that FPU provides a measure of cellulase activity, but additional enzyme activities may be usefully included in an effective mixture of cellulytic enzymes, including but not limited to hemicellulase enzyme activities.

At INBICON™ laboratories, Fredericia, Denmark, we measured as approximately 60 (FPU)/ml the cellulase activity in filter paper units of the commercial cellulase preparation optimized for lignocellulosic biomass hydrolysis and sold by GENENCOR™ under the trademark ACCELLERASE 1500™. The optimization range suggested by GENENCOR™ can thus be recalculated as between about 3 to 15 FPU/g DM pretreated biomass. We measured as approximately 120 FPU/g enzyme the cellulase activity in filter paper units of the commercial cellulase preparation optimized for lignocellulosic biomass hydrolysis and sold by NOVOZYMES™ under the trademark CELLIC CTEC2™. The dosage “target for commercially feasible cellulose hydrolysis” suggested by NOVOZYMES™ can thus be recalculated as between about 1 and 4 FPU/g DM.

The art has previously sought to minimize cellulase dosage, considering that this was critical to commercial feasibility of second generation bioethanol production. See for example ref. 9.

We have discovered that, to the contrary, surprisingly, using SHF or SSF, greater overall process advantage can be obtained using high initial enzyme loadings, at least 15 FPU/g DM.

In some embodiments, the invention provides a high enzyme dose recycling scheme whereby cellulase activity levels of at least 15 FPU/g DM, or at least 12 FPU/g DM, or at least 10 FPU/g DM are maintained over multiple hydrolysis cycles. A high enzyme dose need be added only in an initial hydrolysis cycles. High enzyme levels are maintained in subsequent hydrolysis rounds by recovery of cellulase activity from a previous cycle and supplementation with a comparatively low dose of fresh cellulase enzyme preparation.

The high dose system provides overall advantage even where consumption of enzyme per liter ethanol is increased relative to a low dose system. The use of a high dose regime results in dramatic reduction of hydrolysis times. This reduction in hydrolysis times provides direct benefits in production scale in that capital costs are reduced (smaller hydrolysis tanks can be used) and production capacity increased (higher biomass throughput is achieved). Further, in the high dose regime, lignocellulosic biomass is more completely hydrolysed such that conversion approaches 100%. This in turn reduces overall biomass costs per liter ethanol. In other cases, hydrolysis yields can be so significantly improved and cellulase activity recycled to so high degree that the high dose regime provides equivalent or even lower final enzyme consumption per liter ethanol than can be achieved using lower enzyme doses recommended by commercial enzyme suppliers.

Table 1 shows calculated values of a theoretical high initial cellulase dose that can be approximately maintained over multiple hydrolysis cycles using methods of the invention, with enzyme cost per liter ethanol equivalent to a low dose regime. As shown, the level of cellulase activity recovery defines the level of high enzyme dose that can be sustained through low dose supplementation at each hydrolysis cycle. The supplementation close as shown is simply [1−(% recovery/100)]*(enzyme dose required to achieve full conversion). The theoretical high dose calculation is based on the assumption of 100% conversion in the hydrolysis. The theoretical sustainable high dose should thus be corrected by the factor (actual conversion at high dose)/100%. Equivalent enzyme cost means that the total enzyme consumption per liter ethanol produced at full conversion over 10 cycles of recycling with supplementation is equivalent to the total enzyme consumption per liter ethanol produced over 10 cycles of ordinary fermentation at 5 FPU/g DM at equivalent DM. “Ordinary fermentation” refers to a standard condition of 6 hours prehydrolysis of steam pretreated wheat straw at 25% insoluble fiber using 5 FPU/g DM of a commercial cellulase preparation optimized for hydrolysis of lignocellulosic biomass and provided by NOVOZYMES™ under the trademark CELLIC CTEC2™ at 50° C. followed by 144 hours SSF at 30-33° C. using common bakers' yeast.

The table 1 calculation is determined as follows: The equivalent enzyme cost per liter ethanol target is the typical ethanol yield at 5 FPU/g DM in the SSF regime 144 hours, 25% DM. This typical yield is 70% theoretical conversion, which yield 152 liters ethanol at the reference level of DM.

The equivalence target is thus 5 FPU/152 liters ethanol or 0.0328947 FPU/I ethanol, where g DM is constant.

100% conversion at the reference DM level is 218 liters ethanol. Applying the assumption that complete conversion can be achieved within this SSF regime for any dose over 15 FPU, we calculate total enzyme used over ten cycles of recycling with enzyme supplementation.

We have [total enzyme FPU]/2180 liter ethanol=0.0328947 FPU/liter ethanol, where g DM is constant.

The term for [total enzyme FPU] is a function of both recovery % and starting enzyme concentration, given by X, which can be solved for so as to satisfy the condition [total enzyme]=(2180)(0.0328947)=71.7

The calculation of X is determined as follows: [((9 recycle rounds)*(1−recov % supplementation)+1)*X]=71.7

Thus, for example, the calculation at 15 FPU, with 58% recovery, is: [total enzyme FPU]=15+9*(6.3 supplementation dose)=15+56.7=71.7

Similarly, for example, the calculation at 18.1 FPU, with 67% recovery, is [total enzyme FPU]=18+9*(5.96 supplementation dose)=18.1+53.6=71.7

TABLE 1
Theoretical high cellulase enzyme dose in FPU/g DM sustainable at
equivalent enzyme cost per liter ethanol by given supplementation dose in
FPU/g DM at given average % cellulase activity recovery.

| % recov/ | Theory | Theory |
|---|---|---|
| 100 | High Dose | Suppl. Dose |
| 0.58 | 15 | 6.3 |
| 0.59 | 15.28785 | 6.268017 |
| 0.6 | 15.58696 | 6.234783 |
| 0.61 | 15.898 | 6.200222 |
| 0.62 | 16.22172 | 6.164253 |
| 0.63 | 16.55889 | 6.12679 |
| 0.64 | 16.91038 | 6.087736 |
| 0.65 | 17.27711 | 6.046988 |
| 0.66 | 17.6601 | 6.004433 |
| 0.67 | 18.06045 | 5.95995 |
| 0.68 | 18.47938 | 5.913402 |
| 0.69 | 18.91821 | 5.864644 |
| 0.7 | 19.37838 | 5.813514 |
| 0.71 | 19.8615 | 5.759834 |
| 0.72 | 20.36932 | 5.703409 |
| 0.73 | 20.90379 | 5.644023 |
| 0.74 | 21.46707 | 5.581437 |
| 0.75 | 22.06154 | 5.515385 |
| 0.76 | 22.68987 | 5.44557 |
| 0.77 | 23.35505 | 5.371661 |
| 0.78 | 24.0604 | 5.293289 |
| 0.79 | 24.80969 | 5.210035 |
| 0.8 | 25.60714 | 5.121429 |
| 0.81 | 26.45756 | 5.026937 |
| 0.82 | 27.36641 | 4.925954 |

In some embodiments, using a high dose regime with a supplementation dose between 1 and 6 FPU/g DM, an enzyme dose of at least 12 FPU/g DM can be sustained over multiple hydrolysis rounds, with enzyme costs per liter ethanol produced equivalent or less than those required by a low dose regime. Enzyme dose may be sustained, meaning that the dose does not drop below the sustain value, or, in some embodiments, it may be maintained on average, meaning that over multiple hydrolysis rounds the dose was, on average, at least the sustain value. In some embodiments, the enzyme dose of at least 12 FPU/g DM is sustained over at least three hydrolysis rounds, or at least four hydrolysis rounds, or at least five hydrolysis rounds, or at least six hydrolysis rounds, or at least seven hydrolysis rounds, or at least 8 hydrolysis rounds, or at least 9 hydrolysis rounds, or at least 10 hydrolysis rounds. In some embodiments, an enzyme dose of at least 12 FPU/g DM is maintained on average over at least three hydrolysis rounds, or at least four hydrolysis rounds, or at least five hydrolysis rounds, or at least six hydrolysis rounds, or at least seven hydrolysis rounds, or at least 8 hydrolysis rounds, or at least 9 hydrolysis rounds, or at least 10 hydrolysis rounds.

In some embodiments, using a high dose regime with a supplementation dose between 1 and 6 FPU/g DM, an enzyme dose of at least 10 FPU/g DM can be sustained over multiple hydrolysis rounds, with enzyme costs per liter ethanol produced equivalent or less than those required by a low dose regime. In some embodiments, the enzyme dose of at least 10 FPU/g DM is sustained over at least three hydrolysis rounds, or at least four hydrolysis rounds, or at least five hydrolysis rounds, or at least six hydrolysis rounds, or at least seven hydrolysis rounds, or at least 8 hydrolysis rounds, or at least 9 hydrolysis rounds, or at least 10 hydrolysis rounds. In some embodiments, an enzyme dose of at least 10 FPU/g DM is maintained on average over at least three hydrolysis rounds, or at least four hydrolysis rounds, or at least five hydrolysis rounds, or at least six hydrolysis rounds, or at least seven hydrolysis rounds, or at least 8 hydrolysis rounds, or at least 9 hydrolysis rounds, or at least 10 hydrolysis rounds.

As used herein, all numerical values of FPU are qualified by “about,” meaning +/−0.1, with rounding to the nearest decimal. For example, 9.86 FPU is 10 FPU as used herein. As used herein all numerical values of % conversion are qualified by “about,” meaning +/0.1, with rounding to the nearest decimal. For example, 94.86% conversion is 95% conversion as used herein. As used herein all numerical values of pretreatment severity or % dry matter content are qualified by “about,” meaning +/0.1, with rounding to the nearest decimal.

Table 2 shows selected examples illustrating schemes that sustain calculated values of enzyme dose at levels of 10 FPU/g DM over ten hydrolysis rounds, at specified levels of cellulase activity recovery, initial enzyme dose in round 1 and supplementation dose. Also shown is the relative enzyme cost per liter ethanol produced in a 72 hour SSF regime compared with a baseline of 5 FPU/g DM at equivalent dry matter in a 144 hour SSF regime, based on an estimated relative ethanol yield at 10 FPU/g DM of 120%.

Table 3 shows selected examples illustrating schemes that sustain calculated values of enzyme dose at levels of 12 FPU/g DM over ten hydrolysis rounds, at specified levels of cellulase activity recovery, initial enzyme dose in round 1 and supplementation dose. Also shown is the relative enzyme cost per liter ethanol produced in a 72 hour SSF regime compared with a baseline of 5 FPU/g DM at equivalent dry matter in a 144 hour SSF regime, based on an estimated relative ethanol yield at 12 FPU/g DM of 129%.

TABLE 2
Selected examples of schemes that sustain 10
FPU/g DM in a high dose recycling regime.

| | enzy | | | comp |
|---|---|---|---|---|
| round | dose | recov % | supp | enz/l |
| 1 | 22 | 0.59 | 4 | 0.966667 |
| 2 | 16.98 | 0.59 | 4 | |
| 3 | 14.0182 | 0.59 | 4 | |
| 4 | 12.27074 | 0.59 | 4 | |
| 5 | 11.23974 | 0.59 | 4 | |
| 6 | 10.63144 | 0.59 | 4 | |
| 7 | 10.27255 | 0.59 | 4 | |
| 8 | 10.06081 | 0.59 | 4 | |
| 9 | 9.935875 | 0.59 | 4 | |
| 10 | 9.862166 | 0.59 | 4 | |
| 1 | 15 | 0.6 | 4 | 0.85 |
| 2 | 13 | 0.6 | 4 | |
| 3 | 11.8 | 0.6 | 4 | |
| 4 | 11.08 | 0.6 | 4 | |
| 5 | 10.648 | 0.6 | 4 | |
| 6 | 10.3888 | 0.6 | 4 | |
| 7 | 10.23328 | 0.6 | 4 | |
| 8 | 10.13997 | 0.6 | 4 | |
| 9 | 10.08398 | 0.6 | 4 | |
| 10 | 10.05039 | 0.6 | 4 | |
| 1 | 15 | 0.61 | 4 | 0.85 |
| 2 | 13.15 | 0.61 | 4 | |
| 3 | 12.0215 | 0.61 | 4 | |
| 4 | 11.33312 | 0.61 | 4 | |
| 5 | 10.9132 | 0.61 | 4 | |
| 6 | 10.65705 | 0.61 | 4 | |
| 7 | 10.5008 | 0.61 | 4 | |
| 8 | 10.40549 | 0.61 | 4 | |
| 9 | 10.34735 | 0.61 | 4 | |
| 10 | 10.31188 | 0.61 | 4 | |
| 1 | 15 | 0.62 | 4 | 0.85 |
| 2 | 13.3 | 0.62 | 4 | |
| 3 | 12.246 | 0.62 | 4 | |
| 4 | 11.59252 | 0.62 | 4 | |
| 5 | 11.18736 | 0.62 | 4 | |
| 6 | 10.93616 | 0.62 | 4 | |
| 7 | 10.78042 | 0.62 | 4 | |
| 8 | 10.68386 | 0.62 | 4 | |
| 9 | 10.62399 | 0.62 | 4 | |
| 10 | 10.58688 | 0.62 | 4 | |
| 1 | 15 | 0.63 | 4 | 0.85 |
| 2 | 13.45 | 0.63 | 4 | |
| 3 | 12.4735 | 0.63 | 4 | |
| 4 | 11.85831 | 0.63 | 4 | |
| 5 | 11.47073 | 0.63 | 4 | |
| 6 | 11.22656 | 0.63 | 4 | |
| 7 | 11.07273 | 0.63 | 4 | |
| 8 | 10.97582 | 0.63 | 4 | |
| 9 | 10.91477 | 0.63 | 4 | |
| 10 | 10.8763 | 0.63 | 4 | |
| 1 | 15 | 0.64 | 4 | 0.85 |
| 2 | 13.6 | 0.64 | 4 | |
| 3 | 12.704 | 0.64 | 4 | |
| 4 | 12.13056 | 0.64 | 4 | |
| 5 | 11.76356 | 0.64 | 4 | |
| 6 | 11.52868 | 0.64 | 4 | |
| 7 | 11.37835 | 0.64 | 4 | |
| 8 | 11.28215 | 0.64 | 4 | |
| 9 | 11.22057 | 0.64 | 4 | |
| 10 | 11.18117 | 0.64 | 4 | |
| 1 | 15 | 0.65 | 4 | 0.85 |
| 2 | 13.75 | 0.65 | 4 | |
| 3 | 12.9375 | 0.65 | 4 | |
| 4 | 12.40938 | 0.65 | 4 | |
| 5 | 12.06609 | 0.65 | 4 | |
| 6 | 11.84296 | 0.65 | 4 | |
| 7 | 11.69792 | 0.65 | 4 | |
| 8 | 11.60365 | 0.65 | 4 | |
| 9 | 11.54237 | 0.65 | 4 | |
| 10 | 11.50254 | 0.65 | 4 | |
| 1 | 26 | 0.68 | 3 | 0.883333 |
| 2 | 20.68 | 0.68 | 3 | |
| 3 | 17.0624 | 0.68 | 3 | |
| 4 | 14.60243 | 0.68 | 3 | |
| 5 | 12.92965 | 0.68 | 3 | |
| 6 | 11.79216 | 0.68 | 3 | |
| 7 | 11.01867 | 0.68 | 3 | |
| 8 | 10.4927 | 0.68 | 3 | |
| 9 | 10.13503 | 0.68 | 3 | |
| 10 | 9.891823 | 0.68 | 3 | |
| 1 | 15 | 0.69 | 3 | 0.7 |
| 2 | 13.35 | 0.69 | 3 | |
| 3 | 12.2115 | 0.69 | 3 | |
| 4 | 11.42594 | 0.69 | 3 | |
| 5 | 10.8839 | 0.69 | 3 | |
| 6 | 10.50989 | 0.69 | 3 | |
| 7 | 10.25182 | 0.69 | 3 | |
| 8 | 10.07376 | 0.69 | 3 | |
| 9 | 9.950893 | 0.69 | 3 | |
| 10 | 9.866116 | 0.69 | 3 | |
| 1 | 15 | 0.7 | 3 | 0.7 |
| 2 | 13.5 | 0.7 | 3 | |
| 3 | 12.45 | 0.7 | 3 | |
| 4 | 11.715 | 0.7 | 3 | |
| 5 | 11.2005 | 0.7 | 3 | |
| 6 | 10.84035 | 0.7 | 3 | |
| 7 | 10.58825 | 0.7 | 3 | |
| 8 | 10.41177 | 0.7 | 3 | |
| 9 | 10.28824 | 0.7 | 3 | |
| 10 | 10.20177 | 0.7 | 3 | |

TABLE 3
Selected examples of schemes that sustain 12 FPU/g
DM in a high dose recycling regime.

| | enzy | recov | | comp |
|---|---|---|---|---|
| round | dose | % | supp | enz/l |
| 1 | 15 | 0.58 | 5 | 0.930233 |
| 2 | 13.7 | 0.58 | 5 | |
| 3 | 12.946 | 0.58 | 5 | |
| 4 | 12.50868 | 0.58 | 5 | |
| 5 | 12.25503 | 0.58 | 5 | |
| 6 | 12.10792 | 0.58 | 5 | |
| 7 | 12.02259 | 0.58 | 5 | |
| 8 | 11.9731 | 0.58 | 5 | |
| 9 | 11.9444 | 0.58 | 5 | |
| 10 | 11.92775 | 0.58 | 5 | |
| 1 | 15 | 0.59 | 5 | 0.930233 |
| 2 | 13.85 | 0.59 | 5 | |
| 3 | 13.1715 | 0.59 | 5 | |
| 4 | 12.77119 | 0.59 | 5 | |
| 5 | 12.535 | 0.59 | 5 | |
| 6 | 12.39565 | 0.59 | 5 | |
| 7 | 12.31343 | 0.59 | 5 | |
| 8 | 12.26493 | 0.59 | 5 | |
| 9 | 12.23631 | 0.59 | 5 | |
| 10 | 12.21942 | 0.59 | 5 | |
| 1 | 15 | 0.6 | 5 | 0.930233 |
| 2 | 14 | 0.6 | 5 | |
| 3 | 13.4 | 0.6 | 5 | |
| 4 | 13.04 | 0.6 | 5 | |
| 5 | 12.824 | 0.6 | 5 | |
| 6 | 12.6944 | 0.6 | 5 | |
| 7 | 12.61664 | 0.6 | 5 | |
| 8 | 12.56998 | 0.6 | 5 | |
| 9 | 12.54199 | 0.6 | 5 | |
| 10 | 12.52519 | 0.6 | 5 | |
| 1 | 15 | 0.61 | 5 | 0.930233 |
| 2 | 14.15 | 0.61 | 5 | |
| 3 | 13.6315 | 0.61 | 5 | |
| 4 | 13.31522 | 0.61 | 5 | |
| 5 | 13.12228 | 0.61 | 5 | |
| 6 | 13.00459 | 0.61 | 5 | |
| 7 | 12.9328 | 0.61 | 5 | |
| 8 | 12.88901 | 0.61 | 5 | |
| 9 | 12.8623 | 0.61 | 5 | |
| 10 | 12.846 | 0.61 | 5 | |
| 1 | 15 | 0.62 | 5 | 0.930233 |
| 2 | 14.3 | 0.62 | 5 | |
| 3 | 13.866 | 0.62 | 5 | |
| 4 | 13.59692 | 0.62 | 5 | |
| 5 | 13.43009 | 0.62 | 5 | |
| 6 | 13.32666 | 0.62 | 5 | |
| 7 | 13.26253 | 0.62 | 5 | |
| 8 | 13.22277 | 0.62 | 5 | |
| 9 | 13.19812 | 0.62 | 5 | |
| 10 | 13.18283 | 0.62 | 5 | |
| 1 | 15 | 0.63 | 5 | 0.930233 |
| 2 | 14.45 | 0.63 | 5 | |
| 3 | 14.1035 | 0.63 | 5 | |
| 4 | 13.88521 | 0.63 | 5 | |
| 5 | 13.74768 | 0.63 | 5 | |
| 6 | 13.66104 | 0.63 | 5 | |
| 7 | 13.60645 | 0.63 | 5 | |
| 8 | 13.57207 | 0.63 | 5 | |
| 9 | 13.5504 | 0.63 | 5 | |
| 10 | 13.53675 | 0.63 | 5 | |
| 1 | 15 | 0.64 | 5 | 0.930233 |
| 2 | 14.6 | 0.64 | 5 | |
| 3 | 14.344 | 0.64 | 5 | |
| 4 | 14.18016 | 0.64 | 5 | |
| 5 | 14.0753 | 0.64 | 5 | |
| 6 | 14.00819 | 0.64 | 5 | |
| 7 | 13.96524 | 0.64 | 5 | |
| 8 | 13.93776 | 0.64 | 5 | |
| 9 | 13.92016 | 0.64 | 5 | |
| 10 | 13.9089 | 0.64 | 5 | |
| 1 | 15 | 0.65 | 5 | 0.930233 |
| 2 | 14.75 | 0.65 | 5 | |
| 3 | 14.5875 | 0.65 | 5 | |
| 4 | 14.48188 | 0.65 | 5 | |
| 5 | 14.41322 | 0.65 | 5 | |
| 6 | 14.36859 | 0.65 | 5 | |
| 7 | 14.33958 | 0.65 | 5 | |
| 8 | 14.32073 | 0.65 | 5 | |
| 9 | 14.30847 | 0.65 | 5 | |
| 10 | 14.30051 | 0.65 | 5 | |
| 1 | 18 | 0.66 | 4 | 0.837209 |
| 2 | 15.88 | 0.66 | 4 | |
| 3 | 14.4808 | 0.66 | 4 | |
| 4 | 13.55733 | 0.66 | 4 | |
| 5 | 12.94784 | 0.66 | 4 | |
| 6 | 12.54557 | 0.66 | 4 | |
| 7 | 12.28008 | 0.66 | 4 | |
| 8 | 12.10485 | 0.66 | 4 | |
| 9 | 11.9892 | 0.66 | 4 | |
| 10 | 11.91287 | 0.66 | 4 | |
| 1 | 15 | 0.67 | 4 | 0.790698 |
| 2 | 14.05 | 0.67 | 4 | |
| 3 | 13.4135 | 0.67 | 4 | |
| 4 | 12.98705 | 0.67 | 4 | |
| 5 | 12.70132 | 0.67 | 4 | |
| 6 | 12.50988 | 0.67 | 4 | |
| 7 | 12.38162 | 0.67 | 4 | |
| 8 | 12.29569 | 0.67 | 4 | |
| 9 | 12.23811 | 0.67 | 4 | |
| 10 | 12.19953 | 0.67 | 4 | |
| 1 | 15 | 0.68 | 4 | 0.790698 |
| 2 | 14.2 | 0.68 | 4 | |
| 3 | 13.656 | 0.68 | 4 | |
| 4 | 13.28608 | 0.68 | 4 | |
| 5 | 13.03453 | 0.68 | 4 | |
| 6 | 12.86348 | 0.68 | 4 | |
| 7 | 12.74717 | 0.68 | 4 | |
| 8 | 12.66807 | 0.68 | 4 | |
| 9 | 12.61429 | 0.68 | 4 | |
| 10 | 12.57772 | 0.68 | 4 | |
| 1 | 15 | 0.69 | 4 | 0.790698 |
| 2 | 14.35 | 0.69 | 4 | |
| 3 | 13.9015 | 0.69 | 4 | |
| 4 | 13.59204 | 0.69 | 4 | |
| 5 | 13.3785 | 0.69 | 4 | |
| 6 | 13.23117 | 0.69 | 4 | |
| 7 | 13.12951 | 0.69 | 4 | |
| 8 | 13.05936 | 0.69 | 4 | |
| 9 | 13.01096 | 0.69 | 4 | |
| 10 | 12.97756 | 0.69 | 4 | |

In some embodiments the invention provides a method of processing lignocellulosic biomass comprising
Providing hydrothermally pretreated lignocellulosic biomass
Subjecting said pretreated biomass to an initial enzymatic hydrolysis using an initial cellulase enzyme dose of at least 15 FPU/g DM to a conversion of about 95% or more, followed by
Subsequent hydrolysis cycles wherein cellulase activity recovered from one hydrolysis mixture is used along with a fresh cellulase supplementation dose of between 1-6 FPU/g DM to hydrolyse additional biomass in a subsequent hydrolysis mixture,
   wherein the amount of cellulase activity recovered from one hydrolysis mixture and reused in a subsequent hydrolysis mixture is on average at least about 58% of the amount present at the start of the hydrolysis from which activity is recovered, and wherein the cycle of enzyme recovery and supplementation with fresh cellulase in a subsequent hydrolysis mixture is repeated three or more times.

The term “hydrothermally pretreated” as used herein refers to material pretreated by heating to high temperatures with liquid water and/or steam, optionally including addition of acids, bases or other chemicals. Steam pretreatment typically may be conducted either as a “steam explosion” or using high pressure steam without explosive release of pretreated biomass. Steam pretreatment is typically conducted at high temperatures, between 170 and 220° C., and at high pressures, between 4 and 20 bar, where water exists as a mixture of liquid and vapour. In some embodiments, lignocellulosic biomass is pretreated by hydrothermal pretreatment at temperatures between 170 and 200° C. and at lower severity, <20% of the lignin content of the feedstock is transferred to the liquid phase. For example, in some embodiments, biomass is pretreated to log severity less than 3.9. In some embodiments, fiber fraction is obtained from hydrothermally pretreated biomass by pressing so as to separate fiber fraction from liquid fraction or simply to remove excess liquid from fibers, where pretreatment is conducted by steam explosion. Alternatively, any of a variety of hydrothermal pretreatment methods known in the art may be used, including dilute acid pretreatment, pretreatment with ammonia or base catalyst addition, or other methods. Hydrothermal pretreatments conducted in the pH range 2.5 to 8 are typically termed “autohydrolysis” treatments, since these do not rely on added acid or base catalyst. Autohydrolysis requires somewhat higher temperatures, but avoids requirement for added industrial chemicals.

Any suitable lignocellulosic biomass pretreated by hydrothermal methods well known in the art may be used to practice embodiments of the invention. Some embodiments are practiced using lignocellulosic feedstocks including wheat straw, rice straw, bagasse, grasses, corn stover, or empty fruit bunches.

The term “fiber fraction” as used herein refers to insoluble material that is recovered from hydrothermally pretreated biomass after removal of excess liquid comprising dissolved sugars and other soluble products of pretreatment. The fibers of fiber fraction are swelled with associated aqueous content. As will be readily understood by one skilled in the art, any quantity of fiber fraction may be used including any portion of a total mass of pretreated material, which is typically accumulated and consumed continuously in production scale processing.

In some embodiments, the total % dry matter content used in initial hydrolysis is, on average, equivalent, or within +/−10%, of the % dry matter content used in subsequent hydrolysis cycles.

Hydrothermally pretreated lignocellulosic biomass may be obtained by methods well known in the art, including but not limited to methods of pretreatment and processing described in ref. 14, Petersen et al., “Optimization of hydrothermal pretreatment of wheat straw for production of bioethanol at low water consumption without addition of chemicals,” Biomass and Bioenergy (2009) 33:834-840, which is hereby expressly incorporated by reference in entirety.

In some embodiments, the pretreated biomass is divided into an insoluble fiber fraction, comprising primarily lignin and cellulose, and a liquid fraction. Typically, in order to achieve the very high enzyme concentrations used in some embodiments of the invention, the dry matter subject to hydrolysis is diluted with a solution of recycled enzymes. Because of this dilution effect, the dry matter content of the fiber fraction is preferably brought to a high level. For example, where hydrolysis is conducted at some high dry matter >20%, the fiber fraction is preferably brought to >30% DM, in order to make a hydrolysis mixture having dry matter content >20% after recycled enzymes are added. The fiber fraction is then subject to enzymatic hydrolysis using a high initial cellulase loading, preferably at least 15 FPU/g DM.

In some embodiments, enzymatic hydrolysis may be conducted using fiber fraction. In some embodiments, enzymatic hydrolysis may be conducted using pretreated biomass that comprises both insoluble solids and also liquid comprising solubilized sugars and other soluble products of pretreatment. For example, when biomass is pretreated by steam explosion, both liquid and fiber fraction are obtained in a mixture that can be used for enzymatic hydrolysis in some embodiments.

In some embodiments, enzymatic hydrolysis may be conducted at dry matter at least 10%, or at least 11%, or at least 12%, or at least 13%, or at least 14%, or at least 15%, or at least 16%, or at least 17%, or at least 18%, or at least 19%, or at least 20% at the start of hydrolysis.

At high enzyme loading, the fiber fraction in high dry matter hydrolysis (>20%) can, under conditions optimal for cellulase activity, be hydrolysed to a pumpable liquid within a short time, preferably less than 8 hours, or less than 6 hours, or less than 4 hours, or less than 2 hours. Alternatively the fiber fraction can be rapidly hydrolysed to complete conversion.

Enzymatic hydrolysis of fiber fraction or of a slurry comprising both insoluble solids and also both insoluble solids and also liquid comprising solubilized sugars and other soluble products of pretreatment may be conducted either as an SHF process, where biomass is hydrolysed to fermentable sugars that are subsequently fermented, or as an SSF process, where biomass is hydrolysed to fermentable sugars concurrently with fermentation, or by some combination, for example, SSF in initial hydrolysis followed by subsequent steps of SHF. In SSF, at initial dry matter content >20%, biomass is typically subject to pre-hydrolysis so as to liquefy the fiber fraction, followed by addition of fermentive organisms. Accordingly, the term “hydrolysis mixture” as used herein may refer to either an SHF mixture or to an SSF fermentation. In some embodiments, hydrolysis is conducted at high initial enzyme loading (at least 15 FPU/g DM biomass) followed by fermentation to at least 4% ethanol by weight either in an SHF process or by prehydrolysis followed by SSF or in an SSF process. In some embodiments, the fermentable sugars released by enzymatic hydrolysis are further fermented to ethanol.

In some embodiments, pretreated biomass is hydrolysed using at least 15, 16, 17, or 18 FPU/g DM initial cellulase dose. Hydrolysis may also be conducted using at least 20 FPU/g DM, or at least 22 FPU/g DM, or at least 24 FPU/g DM, or at least 26 FPU/g DM initial dose.

In some embodiments, hydrolysis is conducted on a commercial scale, involving at least 40 kg pretreated biomass, or at least 100 kg, or at least 500 kg, or at least 1,000 kg, or at least 5,000 kg.

The term “conversion” as used herein refers, in an SSF process, to conversion of cellulose into ethanol, and in an SHF process, to conversion of cellulose into glucose. The term “% conversion” refers to % of the amount that could theoretically be obtained based on the cellulose content of the material. 100% theoretical recovery of glucose from cellulose is 1.110 g glucose per g cellulose. 100% theoretical recovery of ethanol from glucose is 0.510 g ethanol per g glucose or from cellulose 0.459 g ethanol per g cellulose.

The term “dry matter” as used herein refers to insoluble solids.

In some embodiments, enzymatic hydrolysis is conducted at a high initial percentage of dry matter, >20% at the start of hydrolysis. In embodiments practiced at high dry matter content, >20%, pretreated feedstock is preferably hydrolysed to a pumpable liquid according to the methods described in WO2006/056838, which is hereby expressly incorporated by reference in entirety.

In high dry matter processing, >20%, pretreated lignocellulosic biomass has often been subject to pre-hydrolysis followed by simultaneous saccharification and fermentation (SSF). At least in the case of yeast fermentations, the temperature at which SSF is conducted has typically represented a compromise between optimal conditions for cellulase activity (50° C.) compared with yeast growth (32° C.). Using high cellulase loading, pre-hydrolysis of fiber fraction provides more complete initial hydrolysis. Where hydrolysis is conducted at high dry matter content, for example, >20%, biomass can be liquefied to a pumpable liquid within a very short time,—typically less than 8 hours, or less than 6 hours, or less than 4 hours, or less than 3 hours. In embodiments that rely on yeast fermentation, the liquefied biomass can then be pumped to a separate vessel for SSF under yeast-optimal conditions that are not harmful to cellulase activity. In SSF embodiments, the liquefied material is then preferably pumped to a separate fermentation vessel, where fermentation/SSF proceeds, in some embodiments, under yeast-optimal temperature and pH conditions. High dry matter hydrolysis may also be conducted as an SHF process. Either SSF or SHF fermentation is preferably continued until an ethanol concentration of at least 4% by weight is achieved. Alternatively, other fermentive organisms than yeast can be used in an SSF process.

It will be readily understood that, in practicing embodiments of the invention, the initial hydrolysis mixture provides the first hydrolysis mixture from which cellulase activity is recovered for use in subsequent hydrolysis mixtures.

In some embodiments, the conversion achieved in subsequent hydrolysis cycles after the initial hydrolysis is maintained at greater than 90%, on average, or more preferably, greater than 91%, on average, or more preferably, greater than 92%, on average, or more preferably, greater than 93%, on average, or more preferably, greater than 94%, on average, or more preferably, greater than 95%, on average.

As will be readily understood by one skilled in the art, the term “on average” as used in the expression “wherein the amount of cellulase activity recovered from one hydrolysis mixture and reused in a subsequent hydrolysis mixture is on average at least about 58% of the amount present at the start of the hydrolysis from which activity is recovered” and in reference to maintenance of conversion over subsequent hydrolysis cycles and in other expressions of a similar nature refers to an average taken over any number of hydrolysis cycles, preferably three, or four, or five, or six, or seven, or eight, or nine, or ten. In some embodiments, the amount of cellulase activity recovered from one hydrolysis mixture and reused in a subsequent hydrolysis mixture is on average at least about 58%, or at least about 60%, or at least about 61%, or at least about 62%, or at least about 63%, or at least about 64%, or at least about 65%, or at least about 66%, or at least about 67%, or at least about 68%, or at least about 69%, or at least about 70%.

Cellulase activity can be recovered from one hydrolysis mixture and used in a subsequent hydrolysis mixture by a variety of methods, including methods well known in the art. Cellulase activity may be recovered from the hydrolysis mixture by recovery after enzymatic hydrolysis, in an SHF process, or after fermentation in either an SSF or SHF process, either before or after distillation to recover ethanol. As is well known in the art, cellulase activity can be recovered both in aqueous phase and also from insoluble residual material remaining after hydrolysis in an SHF process or after fermentation in either an SSF or SHF process, either before or after distillation to recover ethanol. The term “hydrolysis residual” as used herein refers to the solid fraction remaining after hydrolysis in an SHF process or after fermentation in either an SSF or SHF process, either before or after distillation to recover ethanol, with pretreated lignocellulosic biomass that has been hydrolysed to about 70% or greater conversion. Cellulase activity can be recovered after hydrolysis or after fermentation but prior to distillation using decanting and ultrafiltration of SSF or SHF hydrolysis mixtures or by other methods known in the art. Additional cellulase activity can be recovered from the solid fraction remaining after SSF or SHF. Cellulase activity can be recycled from the liquid fraction remaining after distillation and from a wash of the solid fraction remaining after distillation in an SSF process.

Recovery of cellulase activity from the hydrolysis mixture refers to recovery after hydrolysis in an SHF process or after fermentation in an either an SHF or SSF process, either before or after distillation to recover ethanol.

In some embodiments, cellulase activity may be recovered from the hydrolysis mixture prior to fermentation, after fermentation prior to distillation, or after vacuum distillation to recover ethanol, with separation of the hydrolysis mixture or distillation bottom product into a solid fraction and a liquid fraction. Cellulase activity may be effectively recovered after vacuum distillation, where low pressure conditions permit ethanol distillation at a lower temperature, typically about 60° C., at which cellulase activity is not appreciably degraded.

The liquid fraction remaining after distillation may be used directly as aqueous content in subsequent hydrolysis cycles. Alternatively, enzyme activity can be recovered from the liquid fraction remaining after distillation or from the liquid fraction obtained after hydrolysis in an SHF process or after fermentation in either an SHF or SSF process by ultra filtration or other methods known in the art including, for example, the methods described in U.S. Pat. No. 4,840,904 and U.S. Pat. No. 4,746,611, which are hereby expressly incorporated by reference in entirety.

Enzyme activity recovered from a liquid fraction remaining after distillation can be used directly as aqueous content in subsequent hydrolysis cycles or can be adsorbed to pretreated biomass fiber fraction in a soaking step, preferably followed by pressing to increase dry matter content.

Cellulase activity may also be recovered in part from the solid fraction remaining after vacuum distillation or from the solid fraction obtained after hydrolysis in an SHF process or after fermentation in either an SHF or SSF process using a variety of methods known in the art, including, for example, by treatment with solutions enriched in surfactants and counter-binding non-specific proteins, such as bovine serum proteins, that displace some lignin-bound enzyme activity, or by any of the methods described in ref. 8, L. Clesceri, et al., “Recycle of the cellulase-enzyme complex after hydrolysis of steam-exploded wood,” Appl. Biochem. and Biotechnol. (1985), 11:433, or in ref. 10, D. Girard and A. Converse, “Recovery of cellulase from lignaceous hydrolysis residue,” Applied Biochem. and Biotechnol. (1993), 39:521, both of which references are hereby expressly incorporated by reference in entirety.

Characteristic enzyme recovery rates will vary depending upon the feedstock used and the method of pretreatment. In general, it appears that recovery of enzyme bound to hydrolysis residual is improved at higher enzyme dose, possibly because non-specific lignin binding becomes saturated.

Characteristic enzyme recovery rates will also vary depending upon the enzyme preparation used. Any suitable cellulase preparation may be used to practice embodiments of the invention. As is well known in the art, a cellulase preparation effective in enzymatic conversion of lignocellulosic biomass should generally include a mixture of different enzymes, including at least one or more endoglucanase, which introduce nicks in the cellulosic polymer chain thereby exposing reducing ends, one more exoglucanase, which catalyze from reducing and non-reducing ends release of oligosaccharide products from the cellulosic polymer chain, and one or more β-glucosidase, which catalyse hydrolysis of oligosaccharide products to fermentable monosaccharides. All three categories of cellulase enzymes can bind lignin-rich residues that remain after hydrolysis of lignocellulosic feedstocks pretreated hydrothermally, without reliance on chemical methods of delignification. This applies also to β-glucosidases which catalyse reactions in solution, without requirement for productive binding to any cellulosic polymer chain. All three categories of cellulase enzyme can also be recovered from hydrolysis residues, especially where the lignocellulosic substrate has been subject to complete conversion, as is typically achieved at the high cellulase levels utilized in practicing embodiments of the invention. Thus, even if the ratio of different enzymes in the multi-enzyme mixture changes during the course of hydrolysis rounds, supplementation with fresh enzyme using the same enzyme preparation generally ensures that no single component of the enzyme mixture becomes “limiting” to overall hydrolysis of the lignocellulosic material.

In some embodiments, the enzyme preparation used to provide supplementation dose may be a different enzyme preparation from that used to provide the initial dose. Using methods such as those described in ref. 12, T. Vinzant et al., “Fingerprinting Trichoderma ressei hydrolases in a commercial cellulase preparation,” Applied Biochem. and Biotechnol. (2001) 91-93:99-107, which is hereby expressly incorporated by reference in entirety, or even using simple stain-free electrophoresis or other methods well known in the art, one skilled in the art can determine through routine experimentation whether particular components of the cellulase enzyme preparation are differentially recovered or not recovered between hydrolysis cycles relative to other components of the preparation. One skilled in the art can accordingly adjust the composition of the enzyme preparation used to provide the supplementation dose so that the relative proportion of different enzymes comprising the preparation used in initial hydrolysis is roughly maintained over the course of multiple hydrolysis cycles.

Suitable cellulase preparations may be obtained by methods well known in the art from a variety of microorganisms, including aerobic and anaerobic bacteria, white rot fungi, soft rot fungi and anaerobic fungi. As described in ref. 13, R. Singhania et al., “Advancement and comparative profiles in the production technologies using solid-state and submerged fermentation for microbial cellulases,” Enzyme and Microbial Technology (2010) 46:541-549, which is hereby expressly incorporated by reference in entirety, organisms that produce cellulases typically produce a mixture of different enzymes in appropriate proportions so as to be suitable for hydrolysis of lignocellulosic substrates. Some sources of cellulase preparations useful for conversion of lignocellulosic biomass include fungi such as species of Trichoderma, Penicillium, Fusarium, Humicola, Aspergillus and Phanerochaete.

Suitable enzyme preparations that may be used to practice disclosed embodiments include commercially available cellulase preparations optimized for lignocellulosic biomass conversion. The term “commercially available cellulase preparation” refers to a mixture of enzyme activities that is sufficient to provide enzymatic hydrolysis of pretreated lignocellulosic biomass and that comprises cellulase, xylanase and B-glucosidase activities. The term “optimized for lignocellulosic biomass conversion” refers to a product development process in which enzyme mixtures have been selected and modified for the specific purpose of improving hydrolysis yields and/or reducing enzyme consumption in hydrolysis of pretreated lignocellulosic biomass to fermentable sugars. Selection and modification of enzyme mixtures may include genetic engineering techniques, for example such as described in ref. 16 (Zhang et al., 2006) or by other methods known in the art.

Commercially available cellulase preparations optimized for lignocellulosic biomass conversion are typically identified by the manufacturer and/or purveyor as such. These are typically distinct from commercially available cellulase preparations for general use or optimized for use in production of animal feed, food, textiles detergents or in the paper industry.

Any suitable commercially available cellulase preparation optimized for lignocellulosic biomass conversion may be used singly or in combination to practice the disclosed embodiments. Initial enzyme activity of such preparations typically comprises at least exoglucanases, endoglucanases, hemicellulases, and beta glucosidases. Such preparations typically comprise endoglucanase activity such that 1 FPU cellulase activity is associated with at least 31 CMC U endoglucanase activity and further typically comprise beta glucosidase activity such that 1 FPU cellulase activity is associated with at least at least 7 pNPG U beta glucosidase activity. It will be readily understood by one skilled in the art that CMC U refers to carboxymethycellulose units. One CMC U of activity liberates 1 umol of reducing sugars (expressed as glucose equivalents) in one minute under specific assay conditions of 50° C. and pH 4.8. It will be readily understood by one skilled in the art that pNPG U refers to pNPG units. One pNPG U of activity liberates 1 umol of nitrophenol per minute from para-nitrophenyl-B-D-glucopyranoside at 50° C. and pH 4.8. It will be further readily understood by one skilled in the art that FPU of “filter paper units” provides a measure of cellulase activity. As used herein, FPU refers to filter paper units as determined by the method of Adney, B. and Baker, J., Laboratory Analytical Procedure #006, “Measurement of cellulase activity”, Aug. 12, 1996, the USA National Renewable Energy Laboratory (NREL), which is expressly incorporated by reference herein in entirety.

Commercially available cellulase preparations optimized for lignocellulosic biomass conversion and provided by GENENCOR™ may be used to practice disclosed embodiments. One specific example of such a cellulase preparation is sold under the tradename ACCELLERASE 1500™.

Commercially available cellulase preparations optimized for lignocellulosic biomass conversion and provided by NOVOZYMES™ may be used to practice disclosed embodiments. One specific example of such a cellulase preparation is sold under the tradename CELLIC CTEC2™.

It will be readily understood by those of ordinary skill in the art that commercially available cellulase preparations optimized for lignocellulosic biomass conversion may become available in future that are provided as individual components in which cellulytic enzyme activities are provided separately from xylanase and B-glucosidase activities with manufacturers' and/or purveyor's recommendations as to how these should be combined. Combinations of such separately provided preparations either according to manufacturer's recommendations or otherwise may also be construed as collectively forming a “commercially available cellulase preparation.”

In some embodiments, it may be advantageous to supplement enzyme preparations with commercially available B-glucosidase preparations and/or xylanase preparations optimized for lignocellulosic biomass conversion. Commercially available xylanase preparations optimized for lignocellulosic biomass conversion and provided by NOVOZYMES may be used to practice disclosed embodiments. One specific example of such a xylanase preparation is sold under the tradename CELLIC HTEC2™. Commercially available xylanase preparations optimized for lignocellulosic biomass conversion and provided by GENENCOR™ may be used to practice disclosed embodiments. Two specific examples of such a xylanase preparation are sold under the tradename ACCELLERASE XY™ and ACCELLERASE XC™. Commercially available B-glucosidase preparations provided by NOVOZYMES may be used to practice the disclosed embodiments. One specific example is the B-glucosidase preparation sold under the trade name NOVOZYMES 188.

In still other embodiments, it may be advantageous to supplement commercially available cellulase preparations optimized for lignocellulosic biomass conversion with B-glucosidase and/or xylanase preparations obtained from cellulytic microorgranisms. As is well known in the art, B-glucosidase helps reduce product inhibition of cellulose hydrolytic reactions, and, accordingly, it may be advantageous to supplement commercially available cellulase preparations optimized for lignocellulosic biomass with additional B-glucosidase activity. Further, some specific enzyme preparations, prepared by methods known in the art, have been reported to offer advantages as supplements to commercially available cellulase preparations optimized for lignocellulosic biomass conversion. See e.g. ref. 15 (Alvira et al., 2011).

In one some embodiment, methods of the invention are practiced using a commercially available cellulase preparation provided by GENENCOR™ that is optimized for lignocellulosic biomass conversion and that comprises exoglucanases, endoglucanases, hemicellulases, and beta glucosidases and having endoglucanase activity such that 1 FPU cellulase activity is associated with at least 31 CMC U endoglucanase activity and further having beta glucosidase activity such that 1 FPU cellulase activity is associated with at least at least 7 pNPG U beta glucosidase activity, such as, for example, the commercial cellulase preparation sold under the trademark ACCELLERASE 1500™. It will be readily understood by one skilled in the art that CMC U refers to carboxymethycellulose units. One CMC U of activity liberates 1 umol of reducing sugars (expressed as glucose equivalents) in one minute under specific assay conditions of 50° C. and pH 4.8. It will be readily understood by one skilled in the art that pNPG U refers to pNPG units. One pNPG U of activity liberates 1 umol of nitrophenol per minute from para-nitrophenyl-B-D-glucopyranoside at 50° C. and pH 4.8.

In one some embodiment, methods of the invention are practiced using a commercially available cellulase preparation provided by GENENCOR™ that is optimized for lignocellulosic biomass conversion and that comprises exoglucanases, endoglucanases, hemicellulases, and beta glucosidases isolated from genetically modified Trichoderma reesei, such as, for example, the commercial cellulase preparation sold under the trademark ACCELLERASE 1500™.

In one some embodiment, the methods of the invention are practiced using a commercially available cellulase preparation provided by GENENCOR™ that is optimized for lignocellulosic biomass conversion and that comprises exoglucanases, endoglucanases, hemicellulases, and beta glucosidases and having a pH optimum of 5.0 or within 0.5 pH units of 5.0, such as, for example, the commercial cellulase preparation sold under the trademark ACCELLERASE 1500™.

In some embodiments, the same commercial cellulase preparation is used to provide both initial dose and supplementation dose. However, a commercial cellulase preparation may also be used to provide only the initial dose and/or to provide some but not all supplementation doses.

In one some embodiment, the methods of the invention are practiced using a commercially available cellulase preparation provided by NOVOZYMES™ that is optimized for lignocellulosic biomass conversion and that comprises exoglucanases, endoglucanases, hemicellulases, and beta glucosidases and having a pH optimum of 5.0 or within 0.5 pH units of 5.0, such as, for example, the commercial cellulase preparation sold under the trademark CELLIC CTEC2™.

In one some embodiment, methods of the invention are practiced using a commercially available cellulase preparation provided by NOVOZYMES™ that is optimized for lignocellulosic biomass conversion and that comprises exoglucanases, endoglucanases, hemicellulases, and beta glucosidases and having beta glucosidase activity such that 1 FPU cellulase activity is associated with at least at least 7 pNPG U beta glucosidase, such as, for example, the commercial cellulase preparation sold under the trademark CELLIC CTEC2™.

In one some embodiment, methods of the invention are practiced using a commercially available cellulase preparation provided by NOVOZYMES™ that is optimized for lignocellulosic biomass conversion and that comprises exoglucanases, endoglucanases, hemicellulases, and beta glucosidases and having beta glucosidase activity such that 1 FPU cellulase activity is associated with at least at least 20 pNPG U beta glucosidase, such as, for example, the commercial cellulase preparation sold under the trademark CELLIC CTEC2™.

For cellulase enzymes that do bind hydrolysis residual, we have discovered a simple and effective basic approach to recovery of bound activity. Cellulases can be recovered from hydrolysis residual using only a simple wash step. Ref. 11, Xu and H. Chen, “A novel stepwise recovery strategy of cellulase adsorbed to the residual substrate after hydrolysis of steam exploded wheat straw,” Applied Biochem. and Biotechnol. (2007) 143:93, which is hereby expressly incorporated by reference in entirety, reports that a simple wash at pH 4.8 recovers substantial activity from hydrolysis residual using cellulase preparations from Penicillium decumbens. Without wishing to be bound by theory, we think the pH/activity relationship may provide opportunities for bound enzyme recovery by a simple wash step. Using commercially available cellulase preparation provided by GENENCOR™ and sold under the trademark ACCELLERASE 1500™, a simple wash at pH 9.0, which is well within the “inactive” pH range, can recover substantial activity from hydrolysis residual remaining after fermentation in an SSF mixture using pretreated wheat straw. Using commercially available cellulase preparation provided by NOVOZYMES™ and sold under the trademark CELLIC CTEC2™, a simple wash at pH 9.0 can recover substantial activity from hydrolysis residual remaining after fermentation in an SSF mixture using pretreated wheat straw.

In some embodiments, the invention provides a method of recovering cellulase activity bound to hydrolysis residual comprising washing hydrolysis residual at the mildest pH extreme that corresponds to substantially decreased enzyme activity. In specifically some embodiments, the wash is conducted at about pH 9.0, or between about 7.0 and 9.0, or between 7.5 and 9.5, or between 8.5 and 9.5. In some embodiments, the recovered cellulase preparation is inactive in this pH range. In some embodiments, about 40% or more of bound cellulase enzyme is recovered in the wash. In some embodiments, hydrolysis residual may be effectively washed without actually separating the insoluble solid fraction from the liquid volume with which it is associated.

In some embodiments, cellulase activity is recovered from supernatant after hydrolysis and from a wash of hydrolysis residual at between pH 7.5 and 9.5. In some embodiments, hydrothermal pretreatment is conducted as autohydrolysis at between pH 2.5 and 8.0, optionally utilizing acetic acid or other organic acids produced during pretreatment to impregnate biomass prior to pretreatment. In some embodiments, pretreatment is conducted to severity less than 3.95.

In some embodiments, cellulase activity is recovered by a wash of hydrolysis residual at between pH 8.5 and 9.5 combined with recovery of free enzymes remaining in the hydrolysis supernatant.

Typically an effective wash can be achieved using a volume of between 1-3 times excess of the volume of residual washed. In some embodiments, it may be advantageous to wash using as much as 10-times excess volume. Any appropriate buffer system may be used for the wash.

In some embodiments, an initial determination is made by enzyme dose ranging of the minimum quantity of cellulase preparation needed to achieve about 95% or greater conversion (“full” conversion as used herein) within a given SSF or SHF regime for the pretreated feedstock used. Recovery of enzyme activity in the liquid fraction remaining after hydrolysis or after fermentation or after distillation is then determined after SSF or SHF at an enzyme dose sufficient to achieve about 95% or greater conversion within the given SSF or SHF regime. In some embodiments, SSF or SHF is typically conducted for <150 hours, preferably for <120 hours, more preferably for <90 hours, still more preferably for <73 hours, even more preferably for <50 hours.

Recovery of enzyme activity from hydrolysis residual is also determined after SSF or SHF at an enzyme dose sufficient to achieve about 95% or greater conversion within the given SSF or SHF regime. In some embodiments, hydrolysis residual is subject to a simple wash, for example at the mildest pH extreme that corresponds to substantially decreased enzyme activity. More specifically, in some embodiments, hydrolysis residual is subject to wash at about pH 9.0, or between about pH 7.0 and 9.0, or between 8.5 and 9.5, using about 3-fold excess volume wash solution, or alternatively, about a 2-fold excess, or using about an equivalent volume wash solution.

Once characteristic cellulase activity recovery rates are known, it is possible to estimate a high initial cellulase dose that can be approximately sustained with comparative overall advantage through enzyme recycling and supplementation of fresh enzyme on each hydrolysis cycle, according to methods of the invention.

The overall recovery can be calculated as recovery of activity in the liquid fraction+recovery from hydrolysis residual wash. In general, where SSF or SHF has been conducted at high DM (>20%), the activity recovered in the liquid fraction remaining after distillation can be directly re-used without further processing. Alternatively, liquid fraction remaining after distillation can be processed by ultra filtration or other methods known in the art to capture enzyme activity with reduced volume and reduced small-solute content.

The activity recovered from hydrolysis residual wash is preferably further processed. In one some embodiment, the hydrolysis residual wash containing recovered cellulase activity can be used in turn to soak in-coming pretreated biomass prior to hydrolysis. About 70% of cellulase activity recovered in hydrolysis residual wash can be readily adsorbed by fresh pretreated feedstock. In continuous processing, readsorption reaches a steady-state, such that effectively all of the recovered activity can be re-used. For high enzyme dose methods of the present invention to provide comparative advantage, the overall recovery rate should generally be at least about 58% on average. Cellulase activity recovered from washing hydrolysis residual can be up-concentrated and introduced in additional hydrolysis cycles. Alternatively, the hydrolysis residual wash containing recovered cellulase activity can be used in turn to soak pre-treated biomass prior to enzymatic hydrolysis. Considerable quantities of cellulase activity recovered in comparatively dilute solution can be adsorbed to pre-treated biomass by this simple soak step, typically about 70% or more.

It will be readily understood by one skilled in the art that “characteristic enzyme recovery rates” are average values. These can preferably be calculated in a cumulative manner over process runs such that there may be some iterations and routine experimentation to achieve appropriate conditions in a given process arrangement.

In some embodiments the supplementation dose is [1−(% recovery/100)]*(enzyme dose required to achieve full conversion in the hydrolysis regime). In other embodiments, a suitable supplementation dose is typically within the range of about 1-6 FPU/g DM. In some embodiments the supplementation dose is selected so as to maintain an average enzyme dose of at least 10 FPU/g DM or at least 12 FPU/g DM over the course of at least three hydrolysis steps. In some embodiments the supplementation dose is selected so as to sustain an enzyme dose of at least 10 FPU/g DM or at least 12 FPU/g DM over the course of at least three hydrolysis steps.

### Example 1

### Enzyme Dose Ranging to Achieve >95% Conversion in SSF Experiments

The experiment was conducted in a 6-chamber free fall reactor working in principle as the 5-chamber reactor described and used in WO2006/056838. The 5-chamber hydrolysis reactor was designed in order to perform experiments with liquefaction and hydrolysis solid concentrations above 20% DM (WIS). The reactor consists of a horizontally placed drum divided into 6 separate chambers each 24 cm wide and 50 cm in height. A horizontal rotating shaft mounted with three paddles in each chamber is used for mixing/agitation. A 1.1 kW motor is used as drive and the rotational speed is adjustable within the range of 2.5 and 16.5 rpm. The direction of rotation is programmed to shift every second minute between clock and anti-clock wise. A water-filled heating jacket on the outside enables control of the temperature up to 80° C.

The experiments used hydrothermally pretreated wheat straw. Wheat straw cut with an average size of 20-70 mm was wetted with a liquid containing acetic acid (2-8 g/l) to a DM of 25-40% and pretreated by steam at 180-200° C. for 5-15 min. The pretreatment was conducted in the Inbicon pilot plant in Skaerbaek, Denmark. After hydrothermal pretreatment pretreated wheat straw was washed with water and separated in to a fibre fraction and a liquid fraction. The fibre fraction contained more than 90% of the cellulose and lignin and portions of the hemicellulose. The separation was conducted using a screw press.

The chambers of the 6 chamber reactor were filled with about 10 kg pressed pretreated wheat straw (fibre fraction with a cellulose content of app. 55%) and water to give an initial content of 22% (water insoluble solids—“WIS” which is equivalent to DM in this context). The pretreated wheat straw was hydrolyzed at 50° C. and pH 5.0 to 5.3 with seven doses of enzyme: 5, 7, 10, 15, 17.5 and 20 FPU/g DM using a commercially available cellulase preparation optimized for lignocellulosic biomass conversion that is provided by NOVOZYMES™ and sold under the trademark CELLIC CTEC2™. The mixing speed was 6 rpm. After 6 hours liquefaction and hydrolysis (termed “pre-hydrolysis”), simultaneous saccharification and fermentation (SSF) experiments were conducted by lowering the temperature to 33° C. and adding 1 g of dry yeast (THERMOSACC™, provided by ETHANOL TECHNOLOGY™) per kg of initial DM.

PEG6000 (10 g/kg DM) and yeast extract (4 g/kg DM) was added in all the tests. The SSF was allowed to proceed for 3 days. Samples from the broth were analyzed with HPLC for sugars, ethanol, glycerol and small organic acids.

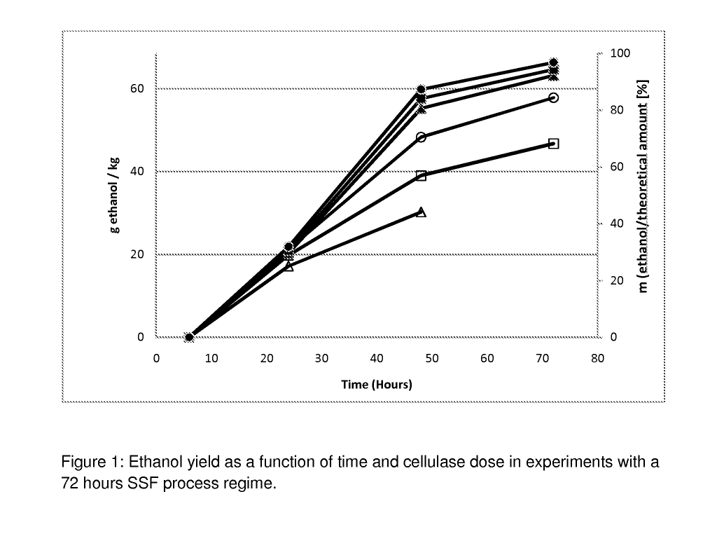

FIG. 1 shows ethanol yield in SSF experiments as a function of time and enzyme dose. The left axis shows the ethanol production as g ethanol/kg total weight while the right axis shows ethanol yield expressed as % theoretical based on the assumption that 51% of the glucose content of cellulose is converted to ethanol. Symbol code: Open triangle 5 FPU/g DM; Open square 7 FPU/g DM; Open circle 10 FPU/g DM; Filled triangle 15 FPU/g DM; Filled square 17.5 FPU/g DM and Filled circle 20 FPU/g DM. Measurements are means of triplicates.

As shown, in order to achieve 95% conversion within the SSF processing regime of 72 hours, an initial enzyme dose of slightly above 17.5 FPU/g DM is some. As will also be readily apparent to one skilled in the art, the processing time required to achieve this very high level of conversion is dramatically reduced at these high cellulase levels. In contrast, final conversion using the low dose regime of 5 FPU/g DM can be achieved only to levels of about 70% after 144 hours of SSF fermentation.

### Example 2

### Standard Activity Curve for Assessing Cellulase Recovery

The experiments used hydrothermally pretreated wheat straw. Wheat straw cut with an average size of 20-70 mm was wetted with a liquid containing acetic acid (2-8 g/l) to a DM of 25-40% and pretreated by steam at 180-200° C. for 5-15 min. The pretreatment was conducted in the in the pilot plant research facility of INBICON™, Skaerbaek, Denmark. After the hydrothermal pretreatment the pretreated wheat straw was washed with water and separated in to a fibre fraction and a liquid fraction. The fibre fraction contained more than 90% of the cellulose and lignin and portions of the hemicellulose. The separation was conducted using a screw press.

The experiment was conducted in shake flasks at 12% DM with total volume 100 ml. The shake flasks were filled with pressed pretreated wheat straw (fibre fraction with a cellulose content of app. 55%) and acetic acid buffer to give an initial content of 12% WIS (which is equivalent with DM in this context). The pretreated wheat straw was hydrolyzed at 50° C. and pH 5.0 to 5.3 with seven doses of enzyme: 7.2, 10.8, 14.4, 18, 21.6 and 25.2 FPU/g DM using a commercially available cellulase preparation optimized for lignocellulosic biomass conversion that is provided by NOVOZYMES™ and sold under the trademark CELLIC CTEC2™.

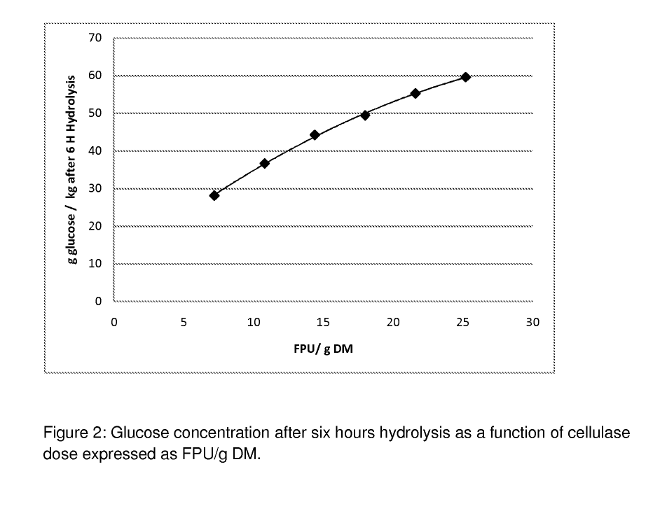

Results are shown in FIG. 2. The graph shows glucose concentration obtained after 6 hours hydrolysis of pretreated wheat straw at different cellulase dose levels. Measurements are means of triplicates. As shown, the relationship between glucose yield obtained at 6 hours hydrolysis and cellulase dose is approximately linear over the range tested. The glucose yield obtained at 6 hours hydrolysis thus provides a standard curve dose response which can be used to estimate cellulase activity recovered in recycling experiments.

### Example 3

### Pre-Hydrolysis and SSF at 18 FPU/g DM Initial Cellulase Dose Followed by Supplementation of Recovered Cellulase Activity Using 6 FPU/g DM Fresh Cellulase Activity Over at Least Three Subsequent Hydrolysis Cycles

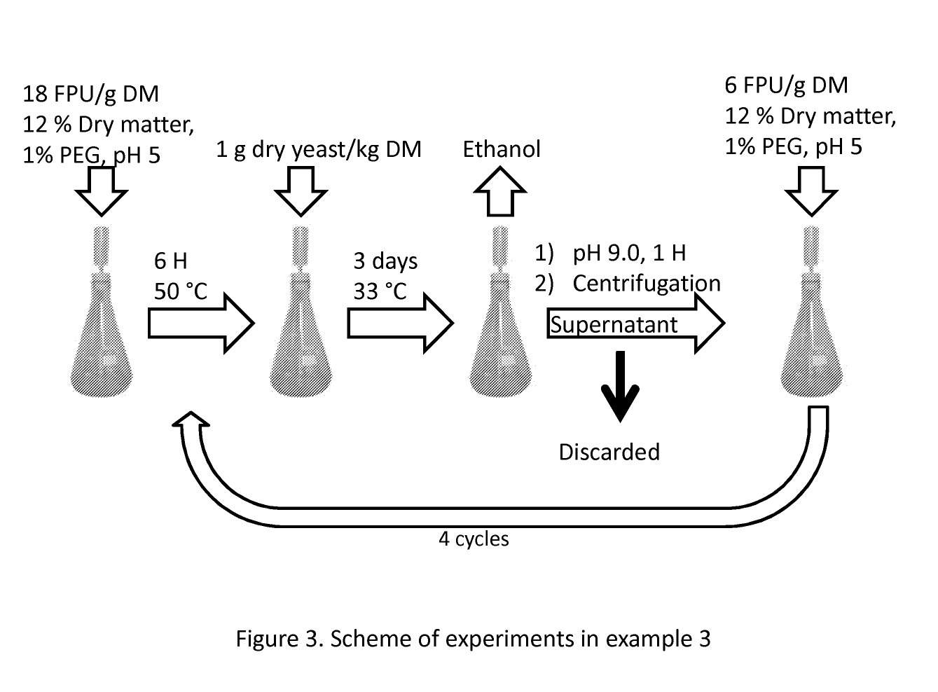

FIG. 3 shows the scheme of experiments in this example 3.

Initial Hydrolysis—Cycle 1.

The experiments used hydrothermally pretreated wheat straw. Wheat straw cut with an average size of 20-70 mm was wetted with a liquid containing acetic acid (2-8 g/l) to a DM of 25-40% and pretreated by steam at 180-200° C. for 5-15 min. The pretreatment was conducted in the pilot plant research facility of INBICON™, Skaerbaek, Denmark. After the hydrothermal pretreatment the pretreated wheat straw was washed with water and separated in to a fibre fraction and a liquid fraction. The fibre fraction contained more than 90% of the cellulose and lignin and portions of the hemicellulose. The separation was conducted using a screw press. The experiment was conducted in shake flasks at 12% DM with total volume 100 ml. The shake flasks were filled with pressed pretreated wheat straw (fibre fraction with a cellulose content of app. 55%) and acetic acid buffer to give an initial content of 12% WIS (which is equivalent with DM in this context). The pretreated wheat straw was hydrolyzed at 50° C. and pH 5.0 to 5.3 with 18 FPU/g DM using a commercially available cellulase preparation optimized for lignocellulosic biomass conversion that is provided by NOVOZYMES™ and sold under the trademark CELLIC CTEC2™ or using a commercially available cellulase preparation optimized for lignocellulosic biomass conversion that is provided by GENENCOR™ and sold under the trademark ACCELLERASE 1500™. The mixing speed was 250 rpm on a shake table.

After 6 hours hydrolysis, glucose measurements were determined.

Simultaneous saccharification and fermentation (SSF) experiments were performed by lowering the temperature to 33° C. after 6 h of liquefaction and hydrolysis (pre-hydrolysis) and adding 1 g of dry yeast (THERMOSACC™, provided by ETHANOL TECHNOLOGY™) per kg of initial DM.

The SSF was allowed to proceed for 3 days. After 3 days, the produced ethanol was evaporated from the shake flasks in a vacuum evaporator at 50° C. The remaining material including both aqueous material and hydrolysis residual was brought to pH 9.0 using 0.25 M NaOH then shaked for 1 hour. The washed residue was centrifuged and the pellet discarded. The washed residue supernatant remaining after centrifugation was pH adjusted to 5.0 and then used directly as buffer in aq subsequent hydrolysis cycle. In some cases, samples from the broth were analyzed with HPLC for sugars, ethanol, glycerol and small organic acids.

Subsequent Hydrolysis—Cycle 2.

Fresh pretreated wheat straw and combined recovered buffer from cycle 1 was mixed and supplemented with 6 FPU/g DM of cellulase preparation then subject to hydrolysis and SSF according to the same procedure as described above for cycle 1. After 6 hours hydrolysis, glucose measurements were determined and used to assess cellulase recovery.

Subsequent hydrolysis—cycle 3.

Fresh pretreated wheat straw and combined recovered buffer from cycle 1 was mixed and supplemented with 6 FPU/g DM of cellulase preparation then subject to hydrolysis and SSF according to the same procedure as described above for cycle 1. After 6 hours hydrolysis, glucose measurements were determined and used to assess cellulase recovery.

Subsequent Hydrolysis—Cycle 4.

Fresh pretreated wheat straw and combined recovered buffer from cycle 1 was mixed and supplemented with 6 FPU/g DM of cellulase preparation then subject to hydrolysis and SSF according to the same procedure as described above for cycle 1. After 6 hours hydrolysis, glucose measurements were determined and used to assess cellulase recovery.

### Subsequent Hydrolysis—Cycle 5.

Fresh pretreated wheat straw and combined recovered buffer from cycle 1 was mixed and supplemented with 6 FPU/g DM of cellulase preparation then subject to hydrolysis and SSF according to the same procedure as described above for cycle 1. After 6 hours hydrolysis, glucose measurements were determined and used to assess cellulase recovery.

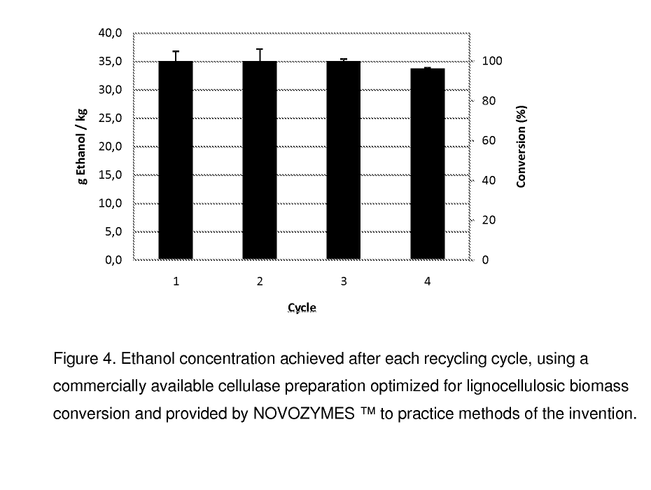

Results obtained using a commercially available cellulase preparation optimized for lignocellulosic biomass conversion that is provided by NOVOZYMES™ and sold under the trademark CELLIC CTEC2™ are shown in FIG. 4. Measurements are means of triplicates. Shown are ethanol concentrations achieved at the end of the 72 hour SSF process regime for an initial hydrolysis and for three subsequent hydrolysis cycles. Ethanol concentrations are expressed as both g/kg total weight and as % theoretical assuming that 51% of glucose content of cellulose is converted to ethanol. As shown, conversions of >95% were achieved in all cases, demonstrating the maintenance of high cellulase levels at least 10 FPU/g DM and at least 12 FPU/g DM over the course of multiple hydrolysis steps using low dose supplementation of recovered cellulases according to methods of the invention.

These results demonstrate effectiveness of methods of the invention, where processing times are halved and conversions increased relative to a low dose regime.

Recovery of cellulase activity between hydrolysis cycles obtained using a commercially available cellulase preparation optimized for lignocellulosic biomass conversion that is provided by NOVOZYMES™ and sold under the trademark CELLIC CTEC2™ are shown in Table 2.

TABLE 2
Calculated cellulase activity recovery based on
measurement of glucose after 6 hours prehydrolysis,
using a commercially available cellulase preparation optimized for
lignocellulosic biomass conversion provided by NOVOZYMES ™.

| | g glucose/ | | FPU g/DM | |
|---|---|---|---|---|
| | kg after 6 H | FPU g/DM | (estimated) − | Recovery |
| Cycles | Hydrolysis | (estimated) | 6 suppl. | (%) |
| 1 | 49.4 | 18.0 | | |
| 2 | 46.7 | 16.1 | 10.1 | 56 |
| 3 | 51.1 | 18.7 | 12.7 | 79 |
| 4 | 58.3 | 24.1 | 18.1 | 97 |

As shown, the glucose concentration measurement after 6 hours hydrolysis obtained in the initial hydrolysis cycle 1 coincides with the level expected for 18 FPU g/DM in applying the standard curve shown in FIG. 2 and explained in Example 2. Cellulase recovery between hydrolysis cycles was estimated as follows. In cycle 2, the FPU estimate obtained by applying the standard curve shown in FIG. 2 was 16.1 FPU g/DM. Subtracting 6 FPU g/DM, which corresponds to the supplementation dose used, the estimated recovery was 10.1 FPU g/DM, which is 56% of the amount present at the start of cycle 1. In cycle 3, the FPU estimate obtained by applying the standard curve shown in FIG. 2 was 18.7 FPU g/DM. Subtracting 6 FPU g/DM, which corresponds to the supplementation dose used, the estimated recovery was 12.7 FPU g/DM, which is 79% of the amount present at the start of cycle 2. In cycle 4, the FPU estimate obtained by applying the standard curve shown in FIG. 2 was 24.1 FPU g/DM. Subtracting 6 FPU g/DM, which corresponds to the supplementation dose used, the estimated recovery was 18.1 FPU g/DM, which is 97% of the amount present at the start of cycle 3. The recovery of cellulase activity on average was 77% over three subsequent hydrolysis cycles wherein cellulase activity recovered from one hydrolysis mixture is used along with a fresh cellulase supplementation dose to hydrolyse additional fiber fraction in a subsequent hydrolysis mixture. This indicates that, as described in Table 1, a high dose of about 18 FPU/g DM can be maintained using methods of the invention with total enzyme cost per liter ethanol produced that is equivalent or lower than that achieved using a low dose regime.

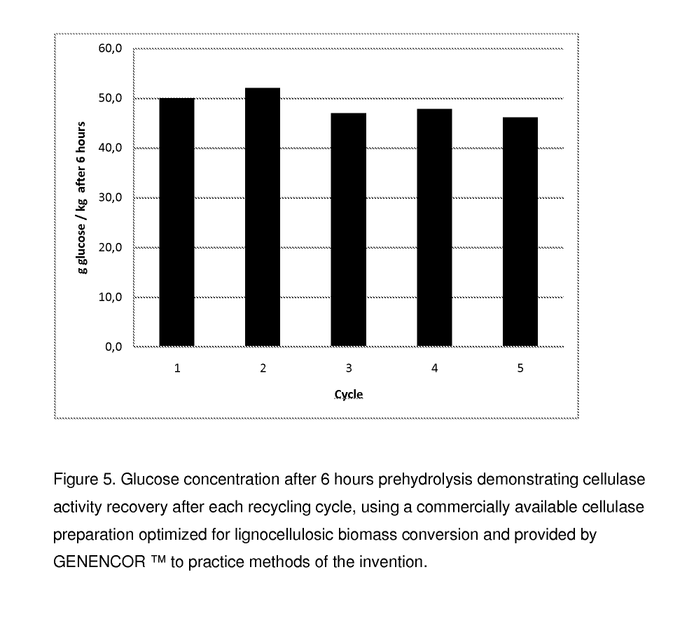

Results obtained using a commercially available cellulase preparation optimized for lignocellulosic biomass conversion that is provided by GENENCOR™ and sold under the trademark ACCELLERASE 1500™ are shown in FIG. 5. Measurements are means of triplicates. Shown are glucose concentrations achieved after 6 hours hydrolysis for an initial hydrolysis and for three subsequent hydrolysis cycles. Ethanol concentrations achieved at the end of the 72 hour SSF process regime were not determined. As shown, the FPU estimate obtained by applying the standard curve shown in FIG. 2 indicates that cellulase levels of above 16 FPU/g DM were sustained for 4 subsequent hydrolysis cycles using methods of the invention. This further demonstrates effectiveness of methods of the invention, where processing times are halved and conversions increased relative to a low dose regime.

Recovery of cellulase activity between hydrolysis cycles obtained using a commercially available cellulase preparation optimized for lignocellulosic biomass conversion that is provided by GENENCOR™ and sold under the trademark ACCELLERASE 1500™ are shown in Table 3.

TABLE 3
Calculated cellulase activity recovery based on
measurement of glucose after 6 hours prehydrolysis,
using a commercially available cellulase preparation optimized for
lignocellulosic biomass conversion provided by GENENCOR ™.

| | g glucose/ | | | |
|---|---|---|---|---|
| | kg after | | FPU g/DM | |
| | 6 H | FPU g/DM | (estimated) − | Recovery |
| Cycles | Hydrolysis | (estimated) | suppl. | (%) |
| 1 | 50.0 | 18.0 | | |
| 2 | 52.0 | 19.3 | 13.3 | 74 |
| 3 | 47.0 | 16.2 | 10.2 | 53 |
| 4 | 47.8 | 16.7 | 10.7 | 66 |
| 5 | 46.1 | 15.7 | 9.7 | 58 |

As shown, the glucose concentration measurement after 6 hours hydrolysis obtained in the initial hydrolysis cycle 1 coincides with the level expected for 18 FPU g/DM in applying the standard curve shown in FIG. 2 and explained in Example 2. Cellulase recovery between hydrolysis cycles was estimated as follows. In cycle 2, the FPU estimate obtained by applying the standard curve shown in FIG. 2 was 19.3 FPU g/DM. Subtracting 6 FPU g/DM, which corresponds to the supplementation dose used, the estimated recovery was 13.3 FPU g/DM, which is 74% of the amount present at the start of cycle 1. In cycle 3, the FPU estimate obtained by applying the standard curve shown in FIG. 2 was 16.2 FPU g/DM. Subtracting 6 FPU g/DM, which corresponds to the supplementation dose used, the estimated recovery was 10.2 FPU g/DM, which is 53% of the amount present at the start of cycle 2. In cycle 4, the FPU estimate obtained by applying the standard curve shown in FIG. 2 was 16.7 FPU g/DM. Subtracting 6 FPU g/DM, which corresponds to the supplementation dose used, the estimated recovery was 10.7 FPU g/DM, which is 66% of the amount present at the start of cycle 3. In cycle 5, the FPU estimate obtained by applying the standard curve shown in FIG. 2 was 15.7 FPU g/DM. Subtracting 6 FPU g/DM, which corresponds to the supplementation dose used, the estimated recovery was 9.7 FPU g/DM, which is 58% of the amount present at the start of cycle 4. The recovery of cellulase activity on average was 63% over four subsequent hydrolysis cycles wherein cellulase activity recovered from one hydrolysis mixture is used along with a fresh cellulase supplementation dose to hydrolyse additional fiber fraction in a subsequent hydrolysis mixture. This indicates that, as described in Table 1, a high dose of about 16 FPU/g DM can be maintained using methods of the invention with total enzyme cost per liter ethanol produced that is equivalent or lower than that achieved using a low dose regime.

The examples and descriptions provide representative examples of particular embodiments and are not intended to limit the scope of the invention as defined by the claims.

The following references are hereby incorporated by reference in entirety.

### REFERENCES

1. D. Lee, et al., “Evaulation of cellulase recycling strategies for the hydrolysis of lignocellulosic substrates.” Biotechnol. and Bioeng. (1995), 45:328.
2. J. Knutsen and R. Davis, “Cellulase retention and sugar removal by membrane ultrafiltration during lignocellulosic biomass hydrolysis.” Appl. Biochem. and Biotechnol. (2004), 113-116:585.
3. D. Gregg and J. Saddler, “Factors affecting cellulose hydrolysis and the potential of enzyme recycle to enhance the efficiency of an integrated wood to ethanol process.” Biotechnol. and Bioeng. (1996), 51:375.
4. M. Tu, et al., “Recycling cellulases during the hydrolysis of steam exploded and ethanol pretreated lodgepole pine.” Biotechnol. Prog. (2007), 23:1130.
5. M. Tu, et al., “Evaluating the distribution of cellulases and the recycling of free cellulases during the hydrolysis of lignocellulosic substrates.” Biotechnol. Prog. (2007), 23:398.
6. W. Sattler, et al., “The effect of enzyme concentration on the rate of the hydrolysis of cellulose.” Biotechnol. and Bioeng. (1989), 33:1221.
7. M. Mandels, et al., “Enzymatic hydrolysis of cellulose: Evaluation of cellulase culture filtrates under use conditions.” Biotechnol. and Bioeng. (1981), 23:2009.
8. L. Clesceri, et al., “Recycle of the cellulase-enzyme complex after hydrolysis of steam-exploded wood.” Appl. Biochem. and Biotechnol. (1985), 11:433.
9. Novozymes grant “Development of a Commercial-Ready Enzyme Application System for Ethanol,” funded by the US Department of Energy in 2008
10. D. Girard and A. Converse, “Recovery of cellulase from lignaceous hydrolysis residue.” Applied Biochem. and Biotechnol. (1993), 39:521.
11. J. Xu and H. Chen, “A novel stepwise recovery strategy of cellulase adsorbed to the residual substrate after hydrolysis of steam exploded wheat straw.” Applied Biochem. and Biotechnol. (2007) 143:93.
12. T. Vinzant et al., “Fingerprinting Trichoderma ressei hydrolases in a commercial cellulase preparation,” Applied Biochem. and Biotechnol. (2001) 91-93:99-107.
13. R. Singhania et al., “Advancement and comparative profiles in the production technologies using solid-state and submerged fermentation for microbial cellulases,” Enzyme and Microbial Technology (2010) 46:541-549.
14. M. Petersen et al., “Optimization of hydrothermal pretreatment of wheat straw for production of bioethanol at low water consumption without addition of chemicals,” Biomass and Bioenergy (2009) 33:834-840.
15. Alvira, Petal., “Pretreatment technologies for an efficient bioethanol production process based on enzymatic hydrolysis: A review,” Bioresource Technology (2010), 101:4851.
16. Zhang, P., et al.,“Outlook for cellulase improvement: Screening and selection strategies,” Biotechnology Advances (2006), 24:452

1. A method of processing lignocellulosic biomass comprising
Providing hydrothermally pretreated lignocellulosic biomass
Subjecting said pretreated biomass to an initial enzymatic hydrolysis using an initial cellulase enzyme dose of at least 15 FPU/g DM to a conversion of 95% or more, followed by
Subsequent hydrolysis cycles wherein cellulase activity recovered from one hydrolysis mixture is used along with a fresh cellulase supplementation dose of between 1-6 FPU/g DM to hydrolyse additional biomass in a subsequent hydrolysis mixture,
wherein cellulase activity is recovered from one hydrolysis mixture from supernatant and from a wash of hydrolysis residual at pH between 7.5 and 9.5 and reused in a subsequent hydrolysis mixture such that the amount of cellulase activity recovered is on average at least 58% of the amount present at the start of the hydrolysis from which activity is recovered, and
wherein the cycle of enzyme recovery and supplementation with fresh cellulase in a subsequent hydrolysis mixture is repeated three or more times.

2. The method of claim 1 wherein the hydrolysis mixture is subsequently subject to fermentation to an ethanol concentration of at least 4% by weight.

3. The method of claim 1 wherein cellulase enzyme activity is recovered after vacuum distillation at a temperature of about 60° C. or less.

4. The method of claim 1 wherein the lignocellulosic biomass is wheat straw.

5. The method of claim 1 wherein recovered cellulase activity is included in subsequent hydrolysis cycles by using recovered enzyme solution to soak fresh pretreated biomass prior to hydrolysis.

6. The method of claim 1 wherein the initial cellulase dose is at least 18 FPU/g DM.

7. The method of claim 1 wherein the cellulase preparation used to provide the supplementation dose is different from the cellulase preparation used to provide the initial cellulase dose.

8. The method of claim 1 wherein the cellulase is a commercially available cellulase preparation that is optimized for lignocellulosic biomass conversion.

9. The method of claim 1 wherein hydrothermal pretreatment is conducted as autohydrolysis between pH 2.5 and 8.0.

10. The method of claim 1 wherein hydrothermal pretreatment is conducted to log severity less than 3.95.

11. The method of claim 1 wherein hydrolysis is conducted as an SSF process.

12. The method of claim 1 wherein hydrolysis is conducted as an SHF process.

13. The method of claim 1 wherein the supplementation dose is selected so as to maintain an enzyme dose of at least 10 FPU/g DM on average over at least three hydrolysis rounds.

14. The method of claim 1 wherein the supplementation dose is selected so as to maintain an enzyme dose of at least 12 FPU/g DM on average over at least three hydrolysis rounds.

15. The method of claim 1 wherein the supplementation dose is selected so as to sustain an enzyme dose of at least 10 FPU/g DM over at least three hydrolysis rounds.

16. The method of claim 1 wherein the supplementation dose is selected so as to sustain an enzyme dose of at least 12 FPU/g DM over at least three hydrolysis rounds.
